(19) United States
(12) Patent Application Publication
(10) Pub. No.: US 2017/0173209 A1
(43) Pub. Date: Jun. 22, 2017

(54) WOUND CLOSURE COMPOSITIONS AND METHOD

(71) Applicant: Rousseau Research, Inc., Palo Alto, CA (US)

(72) Inventor: Joseph D. Russo, Palo Alto, CA (US)

(73) Assignee: Rousseau Research, Inc., Palo Alto, CA (US)

(21) Appl. No.: 15/377,948

(22) Filed: Dec. 13, 2016

Related U.S. Application Data
(60) Provisional application No. 62/269,842, filed on Dec. 18, 2015.

(51) Int. Cl. A61L 24/04; C09J 4/06; A61L 24/00

(52) U.S. Cl. CPC A61L 24/046; A61L 24/0031; C09J 4/06

(57) ABSTRACT

A medical adhesive that bonds well to human tissue while curing in a fast, controllable mariner. In a preferred form, the medical adhesive includes an oligomer, a hydrogel and/or water soluble polymer and a photoinitiator. Preferred oligomers include epoxides, urethanes, polyethers, polyester or a combination thereof. Hydrogels and water soluble polymers aid adhesion to moist surfaces, such as skin tissue, because they are hydrophilic and biodegradable, Preferred hydrogels include polymer hydrogels (PHGs). Suitable water soluble polymers include polyethylene oxide) (PEO) and poly-2-oxazoline. The photoinitiator is used to obtain fast, controllable curing of the adhesive compound. Curing takes place on demand when ultraviolet (UV) light is applied to the medical adhesive. To increase adhesion as well as to control flexibility and toughness, the medical adhesive may also include one or more monomers. Suitable monomers include acrylates and vinyls.

### CROSS-REFERENCE TO RELATED APPLICATIONS

This non-provisional patent application claims priority under 35 U.S.C. §119(e) to U.S. Provisional Application No. 62/269,842, filed Dec. 18, 2015, and entitled “Wound Closure Compositions and Method”, the disclosure of which is hereby incorporated by reference in its entirety for all purposes.

### FIELD OF INVENTION

The present invention relates to improved medical adhesive and sealant compositions as well as their production and use,

### BACKGROUND OF THE INVENTION

The most common types of surgical adhesives are fibrin based adhesives and cyanoacrylates. Fibrin based adhesives are typically a two-component material consisting of fibrinogen and thrombin. In the presence of small amounts of calcium and factor XIII, the thrombin converts fibrinogen into insoluble fibrin, the final stable form of the agent. Fibrin adhesives are the only commercially available FDA approved medical adhesives in clinical use for hemostats and sealants. There are two forms of fibrin sealants, lyophilized powder kits (Artiss, Baxter, Westlake Village, Calif.) and liquid fibrin sealant (Tisseel, Baxter, Westlake Village, Calif.). The room temperature lyophilized powder kits require reconstitution including mixing. The different sizes (2, 4, and 10 mL kits) of mixed frozen fibrin sealant in preloaded syringes require different thawing times which are the fastest in a sterile water bath (33°-37° C.) on the operative field (5, 5, and 12 minutes, respectively). The thawed unopened frozen pouches may be kept for up to two weeks in a refrigerator for prompt use. This fibrin sealant is similar in cost to commercial pooled plasma liquids approved as hemostats (i.e., approximately $50 ml. of final mixed product for this Class III medical device).

As a medical adhesive and sealant, fibrin based adhesives have several disadvantages, The cure time of fibrin based adhesives cannot be precisely controlled by the surgeon. While they are waiting for the fibrin based adhesives to cure, many surgeons use the cure time to manipulate grafts or flaps in order to assure proper placement. After positioning of the graft or flap, gentle pressure is typically applied for 3 minutes to assure proper adherence. No additional manipulation of the graft or flap should then occur to prevent disruption of the fibrin sealant adhesive bonds and, if this occurs, the incidence of seroma formation and drainage volume will increase as the fibrin sealant begins to function as an anti-adhesive. Moreover, the additional time to complete polymerization may allow the liquid fibrin sealant to migrate to the most dependent portions of the wound before sticking, particularly in wounds with large topographic differences. This may result in an irregular final layer of application. Fibrin based adhesives may also present problems with immunogenicity and the risk of blood transmission diseases, such as HIV and BSF.

Cyanoacrylates or “superglues” are monomer and polymer adhesives which are used in industrial, household and medical applications. Included among these adhesives are the 1, 1-disubstituted ethylene monomers and polymers such as the α-cyanoacrylate. Medical applications of 1, 1-disubstituted ethylene adhesive compositions include use as an alternate and an adjunct to surgical sutures and staples in wound closure as well as covering and protecting surface wounds such as lacerations, abrasions, burns, stomatitis, sores and other topical, surface wounds.

Nonetheless, methyl and ethyl cyanoacrylates have been reported to degrade in aqueous media producing formaldehyde, which causes irritation, inflammation and has carcinogenic potential. These deficiencies have been markedly reduced with use of butyl and octyl cyanoacrylates, which have FDA approval as Class II medical devices for topical use only. Histocryl® butyl (Braun, Aesculap Div., Center Valley, Pa.) and Dermabond® octyl (Ethicon, Division of Johnson & Johnson, Somerville, N.J.) are examples of these medical butyl and octyl cyanoacrylates. Dermabond® octyl cyanoacrylate has approximately 90©0 of the worldwide market for cyanoacrylate Class II wound closure adhesive. Costs of Dermabond® octyl cyanoacrylate products are approximately $20/ml. The FDA approvals limit cyanoacrylates to topical wound closure applications. Nonetheless, cyanoacrylates have been used off label in tissue adhesion, to repair blood vessels (Maldonado et al., 2003) and ophthalmology (Duffy et al., 2005; Setlik et al., 2005; Sharma et al., 2003).

A common problem with cyanoacrylate adhesives is that they are difficult to control. In the case of skin bonding, the formulated Dermabond® octyl cyanoacrylates require special applicator devices to speed the cure on skin. The devices separate the monomer from the curative by use of an expensive glass cylinder, plastic body and porous plug head application device. The glass cylinder separates the cyanoacrylate from the curative accelerator containing porous head. in use, the cylinder is crushed, releasing the fluid cyanoacrylate to be pressed through the porous head, co-eluting the cyanoacrylate with the curative accelerator that had been pre-deposited in the porous plug head. Even with the curative accelerator in the porous plug head, cure times are a few minutes long. More importantly, the time to cure is not controlled by the medical professional.

Other medical adhesives are described in Bettinger's U.S. Pat. No. 8,143,042. The Bettinger patent describes biodegradable elastomers that can be used for a variety of applications, such as surgical glues. The elastomers are prepared by crosslinking pre-polymers containing crosslinkable functional groups, such as acrylate groups.

Other medical adhesive options include urethane-based adhesives. These urethane materials may be prepared under the form of pre-polymers (containing free isocyanate groups) and therefore being able to react with amino groups present in the biological molecules establishing adhesion. Polyurethane pre-polymers were first used as biological adhesives in 1959 for the fusion of bone fragments (Heiss et al., 2006). This adhesive, commercially named as Ostamer®, was composed of a pre-polymer and a catalyst which were mixed just before application. However, the crosslinking reaction lasted 25 to 30 minutes and the adhesive reached its maximal strength after 1 or 2 days. For these reasons, the experimental and clinical results proved to be inadequate. Since then, the development of urethane pre-polymers to be applied as bio-adhesives has been an issue studied by different authors (Lipatova, 1986; Sheikh et al., 2001; Ferreira, 2008). Unfortunately, despite the good adhesion results, the curing time is too long for surgical demands. Also, urethane-based materials have been associated with local inflammation, cytotoxicity and poor biocompatibility.

From all polyurethane based adhesives studied, the most developed was KL-3 (Lipatova, 1986). KL-3 is a mixture of an excess of toluene diisocyanate pre-polymer (TDI, mixture of isomers 2,4 and 2,6) with polyoxypropylene glycol and an accelerator of curing, dimethyltric (aminomethyl) phenol. The amount of accelerator predetermines the curing time. Thus, the surgeon can adjust the curing time depending on the surgical situation. The adhesive cures under conditions of a moist area (since it is applied on the surface of an open wound). Along the polymerization process, the reaction with water (present in the moisture of the wound) formed urea groups and released carbon dioxide. This causes foaming and the formation of a fine porous structure in the application surface. The ability of adhesion of this material was evaluated and proved to be similar to cyanoacrylates.

Polyethylene glycol (PEG) hydrogel was allowed to be part of the strict list of materials approved by FDA for several applications, including biomedical applications (Popat et al., 2004). In the field of wound healing, PEG is used as a sealant, in other words, as a suture adjuvant that helps hemostasis in the wound. These products are available commercially under the brands FocalSeal® (Genyzme Biosurgery, Inc., Cambridge, Mass.), CoSeal® (Cohesion Technologies, Deerfield, Ill.) and DuralSeal® (Confluent Surgical, Inc., Waltham, Mass.), among others. Despite the good results, the time taken to prepare and apply the hydrogel is an issue, considering the need of a previous primer application, which limits the use when hemostasis is the priority.

One approach developed in the art to more accurately control the curing time of medical adhesives is to include a photoinitiator in the adhesive which is sensitive to ultraviolet (UV) light. Ultraviolet curable adhesives offer major advantages compared to pre-polymers systems, such as fast-curing rate and control of the polymerization heat evolution. They are particularly useful for application to weakened and diseased tissue (see, Benson, 2002). Kao et al, (1997), for example, prepared UV irradiation curable bioadhesives based on N-vinylpyrrolidone. Although these adhesives provided suitable adhesive strength, the UV induced setting time was approximately 3 minutes, which is usually an unacceptable length of time for surgical applications.

Dentistry is one market where free radical photopolymers have found wide use in fillers, sealant composites and protective coatings. These dental composites are based on a camphor Quinone photoinitiator and a matrix containing methacrylate oligomers and inorganic fillers such as silicon dioxide. Photo curable adhesives (known as engineering adhesives) are also used in the fabrication and production of catheters, hearing aids, surgical masks, medical filters, and blood analysis sensors. Nonetheless, they are unsuitable for use as tissue adhesives because of their lack of tissue adhesion and compatibility with moist surfaces.

Baroli, B. (2006), Photopolymerization of biomaterials: issues and potentialities in drug delivery, tissue engineering and cell encapsulation applications. J. Chem. Technol. Biotechnol., 81: 491-499. Doi: 10.1002/jctb.1468 reveals that photopolymers have also been explored for uses in drug delivery, tissue engineering and cell encapsulation systems. Photopolymerization processes for these applications are being developed to be carried out in vivo or ex vivo. In vivo photopolymerization would provide the advantages of production and implantation with minimal invasive surgeries. Ex vivo photopolymerization would allow for fabrication of complex matrices and versatility of formulation. Although photopolymers show promise for a wide range of new biomedical applications, biocompatibility with photopolymeric materials must still be addressed and developed. Commercially and non-commercially available molecules and macromolecules used as photopolymerizabie monomers and macro-monomers (or “macromers”) have one feature in common—their backbone needs to have a photopolymerizable residue that normally is located at one or at both ends of the molecule.

Ferreira et al. (2008) report the synthesis of urethanes based on polycaprolactonediol (PCL) being easily crosslinked via UV irradiation to be used as a photocrosslinkable biodegradable bioadhesives. PCL is a semi-crystalline, linear biodegradable aliphatic polyester that has been used in several medical applications already approved by the U.S. Food and Drug Administration, Its structure presents several aliphatic ester linkages that can undergo hydrolysis and its products of degradation are either metabolized by being included in the tricarboxylic acid cycle or eliminated by renal secretion. Ferreira modified the polymer with 2-isocyanatoethylmethacrylate (IEMA) to form a rnacromer that was crosslinked via UV irradiation using Irgacure® 2959 (BASF, Florham Park, N.J.) as the photoinitiating agent. Results showed that it took 60 seconds of irradiation before the curing of the polymer was complete and polymer films were obtained, This is a long length of time when speed of wound closure or sealing is required.

Despite all the advances in medical adhesives, there is still a need for a medical adhesive that bonds well to human tissue while curing in a fast, controllable manner. The medical adhesive should have flexibility and toughness, but not cause inflammation or cytotoxicity.

### BRIEF SUMMARY OF THE INVENTION

The present invention provides a low-cost medical adhesive that bonds well to human and animal tissue while curing in a fast, controllable manner. In its most basic preferred form, the medical adhesive of the present invention includes a combination of an oligomer, a hydrogel and/or water soluble polymer and a photoinitiator. Suitable oligomers for the present invention include epoxides, urethanes, polyethers, or polyesters, preferably dimers, trimers and tetramers, each of which provides specific properties to the resulting material. Each of these oligomers is preferably functionalized by an acrylate.

Hydrogels and water soluble polymers aid adhesion to moist surfaces, such as skin tissue, because they are hydrophilic and biodegradable. Preferred hydrogels include polymer hydrogels (PHGs). Suitable water soluble polymers (WSPs) include poly-2-oxazolines and related pseudo-polypeptides (mostly N-substituted polypeptides, so-called polypeptoids), polyethylene glycol, polyethylene oxide, polyvinyl pyrrolidone, low hydrolysis polyvinyl alcohol, carbomer, water soluble chitosan, polyvinylmethylether and other natural or synthetic water soluble polymers. Such water soluble polymers behave as hydrogels in the cured matrix of the adhesive of the present invention insofar as they absorb water but do not dissolve.

The photoinitiator is used to obtain fast, controllable curing of the adhesive compound. Photoinitiators are compounds that, upon radiation of light, decompose into reactive species that activate polymerization. Preferred photoinitiators for use in the present invention are ultraviolet (UV) light photoinitiators including, but not limited to, 1-cyclohexyl phenyl ketone, 2,2-dimethoxy-2-phenylacetophenone (DMPA), 2,4,6-trimethylbenzoyl-diphenyl phosphine oxide, ethyl(2,4,6-trimethylbenzoyl)-phenyl phosphinate, 2-(hydroxyethoxy) phenyl-2-methyl-1-propanone (Irgacure®-2959; D2959; 12959), α-Hydroxyketone, 2-Hydroxy-1-[4-(2-hydroxyethoxy), Irgacure 6510 Irgacures are ™ of BASF), camphor Quinone/amine, where the amine is triethylamine, triethanolamine and ethyl-N,N-dimethylaminobenzoate.

To increase adhesion as well as control the flexibility and toughness of the adhesive, the adhesive formulation of the present invention preferably also includes one or more monomers. These monomers may also serve as solvent and viscosity modifiers. Preferred monomers include acrylates and vinyls.

The medical adhesives of the present invention are suitable for use as an adjunct to surgical sutures and staples in wound closure, covering and protecting surface wounds such as lacerations, minor cuts, abrasions, burns, stomatitis, sores and other surface wounds, sealing ulcers, bonding approximated skin incisions, and bonding/repairing other soft tissue organ and body part wounds or defects. The timing of the immediate curing of these compositions is totally in the control of the medical professional or user (i.e., “curing on demand”). In the preferred embodiment, this curing on demand is done with little or no exothermic heat being produced.

### DETAILED DESCRIPTION OF THE INVENTION

The unique medical adhesive compositions and sealants of the present invention are obtained, in their most basic form, by combining an oligomer with a hydrogel and/or water soluble polymer and a photoinitiator. To increase adhesion, control flexibility, toughness and/or other attributes of the composition, one or more monomers are preferably also included in the adhesive composition. The adhesive/sealant of the present invention can be applied to tissue and cured in a few seconds, typically less than 5 seconds, by exposure to ultraviolet (UV) light. This is curing on demand. The speed of cure is, of course, dependent upon the thickness and area of adhesive applied.

An oligomer is a compound, intermediate between a monomer and a polymer, normally having between five and one hundred monomer units. Oligomerization is a chemical process that converts monomers to macromolecular complexes through a finite degree of polymerization, usually a few monomer units, in contrast to a polymer, where the number of monomers is not limited. In the present invention, oligomers are used to create the backbone of the adhesive composition of the present invention. The oligomers create desirable properties in the adhesive composition, such as flexibility and adhesion, as well as diminishing the amount of exothermic heat produced (as compared with using monomers as the backbone). Like the monomers which can be added to the adhesive composition of the present invention, the preferred oligomers are those that are available for cross-linking. Oligomers that have in situ functionality as hydrogels may also be used to produce in situ polymer hydrogels for the present invention.

Suitable oligomers for the present invention include epoxides, urethanes, polyethers, or polyesters, preferably dimers, trimers and tetramers, with urethane oligomers being most preferred. Each of these oligomers is typically functionalized by an acrylate. An example shown below is an epoxy oligomer that has been functionalized by acrylic acid:

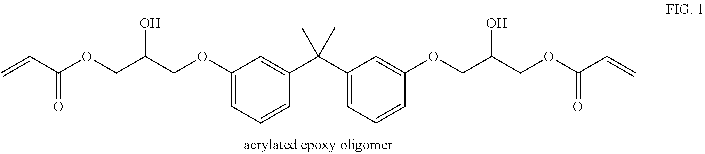 [SMILES: C=CC(=O)OCC(O)COc1cccc(C(C)(C)c2cccc(OCC(O)COC(=O)C=C)c2)c1]

Acrylated epoxy oligomers are useful as coatings on metallic substrates and result in glossy hard coatings. Acrylated urethane oligomers are typically abrasion resistant, tough, and flexible, making ideal coatings for floors, paper, printing plates, and packaging materials. Acrylated polyethers and polyesters result in very hard solvent resistant films. Formulations often are composed of several types of oligomers to achieve the desirable properties for a material.

A monomer is a molecule of low molecular weight capable of reacting with identical or different molecules of low molecular weight to form a polymer, When those monomers react together to form a polymer, the composition “cures” to become an adhesive. The monomers used in the present invention are preferably selected for, among other things, their ability to form photopolymers, to speed curing, increase crosslink density, control viscosity of the adhesive and control mechanical/surface properties. Oligomers, rather than monomers, are preferred for use as the backbone of the adhesive composition of the present invention because rapid polymerization of monomers can be highly exothermic to the point of causing painful burns when such monomer polymerization reactions occur on the surface of human or animal skin. Accordingly, monomers are preferably used in the present invention at lower concentrations as a secondary ingredient. The objective is to create an adhesive that will not burn or cause irritating pain when it is applied to human or animal skin.

Preferred monomers for the present invention are N-vinylpyrrolidone and acrylates. N-vinylpyrrolidone results in a material that is highly flexible when cured and has low toxicity, Acrylates are highly reactive (i.e., allowing for rapid cure rates) and are highly versatile with monomer functionality ranging from monofunctional to tetrafunctional, Other materials which have been studied and may be useful for the present invention are (di)methacrylic or (di)acrylic derivatives of poly(ethylene glycol) (PEG) and its derivatives, poly (ethylene oxide), poly(vinyl alcohol) (PVA) and its derivatives, PEG-polystyrene copolymers (PEG)-(PST), ethylene glycol-lactic acid copolymers (nEGmLA; where n and m are the number of repeat units of EG and LA, respectively), ethylene glycol-lactic acid-caprolactone copolymers (nEGmLAz CL), PLA-b-PEG-b-PLA, PLA-g-PVA poly(D,L-lactide-co-ε-caprolactone), (poly)-anhydrides, anhydrides, urethanes, polysaccharides, dextran collagen, hyaluronic acid, diethyl fumarate/poly(propylene fumarate), and other photopolymerizable residues. A cinnamic derivative of hyaluronic acid has been explored as an injectable, absorbable biomaterial that could be used to prevent postsurgical adhesion formation. Some preferred monomers of the present invention may be used as a precursor to form a hydrogel and/or water soluble polymer.

A photopolymer is a polymer that is formed when one or more oligomers, one or more monomers or a mixture of both is exposed to light, often in the ultraviolet or visible region of the electromagnetic spectrum. These changes are often manifested structurally, for example hardening of the material occurs as a result of cross-linking when exposed to light. Photopolymerizable, bio-use oligorners and/or monomers might produce a semi-degradable, non-degradable linear or crosslinked polymer network. This strongly depends on the type of chemical bonds in the oligomer or monomer backbone. For methacrylic or acrylic monomers, for example, fully degradable networks cannot be produced because the polymerization creates a non-degradable hydrocarbon polymeric backbone chain to which potentially degradable lateral chains are attached. These lateral chains may undergo hydrolytic or enzymatic degradation. Finally, when the degradable chains are eliminated, the polymethacrylic-backbone, depending on its dimensions, might be eventually excreted by glomerular filtration. Glomerular filtration is the process by which the kidneys filter the blood, removing excess wastes and fluids.

Most commonly, photopolymerized systems are typically cured through UV radiation, since ultraviolet light is more energetic. However, the development of dye-based photoinitiator systems have allowed for the use of visible light, having potential advantages of processes that are, perhaps, more simple and safe to handle. UV curing in industrial processes has greatly expanded over the past several decades. Many traditional thermally cured and solvent-based technologies can be replaced by photo polymerization technologies. The advantages of photo polymerization over thermally cured polymerization include high rates of polymerization and environmental benefits from elimination of volatile organic solvents.

A hydrogel is a material made from a water-insoluble polymer that is capable of absorbing a large amount of water (i.e., it is a water swollen polymer network). Their affinity to absorb water is attributed to the hydrophilic nature of the polymeric chains forming the hydrogel structure. Despite their high water absorbing affinity, hydrogels have a great swelling behavior instead of being dissolved in the aqueous surrounding environment as a consequence of the critical crosslinks present in their structure. A good review of hydrogels can be found in Review: Synthetic Polymer Hydrogels for Biomedical Applications, Chemistry and Chemical Technology, Vol. 4, NO. 4, 2010 by Gibas and Janic, the disclosure of which is hereby incorporated by reference in its entirety. Water soluble polymers behave like hydrogels in the adhesive of the present invention by being bound in the adhesive composition's cross-linked matrix.

The preferred hydrogels for use in the present invention are polymer hydrogels (PHGs). These PHGs include, but are not limited to, poly(ethylene glycol) (PEG), poly(oxyethylene) or poly(ethylene oxide) (PEO), hydrogels based on PEG derivatives (e.g., polyethylene glycol methacrylate (PEGMA), polyethylene glycol dimethacrylate (PEGDMA), polyethylene glycol diacrylate (PEGDA), etc.), polyvinyl alcohol (PVA)-based hydrogels, polyvinylpyrrolidone (PVP) based hydrogels, polyimide (PI) hydrogels, polyacrylate (PA) hydrogels, mainly polyacrylamide (PAM), polyurethane (PU) hydrogels, polyethylhexylmethacralate (PEHMA) and polyhydroxyethylmethacrylate (PHEMA). While PHEMA is hydrophobic, when PHEMA is subjected to water it will swell due to the molecule's hydrophilic pendant group. Depending on the physical and chemical structure of the hydrogel polymer, it is capable of absorbing from 10 to 600% water relative to the dry weight. Because of this property, it was one of the first materials to be successfully used in the manufacture of flexible contact lenses. In addition, a di-acrylated pluronic F127 (a poly(ethylene oxide)-b-poly(propylene oxide)-b-poly(ethylene oxide) macromer) has been crosslinked in the presence and absence of vinyl group-modified hyaluronic acid to form hydrogels able to release plasmid DNA. It should be noted that the higher molecular weight polymer hydrogels and water soluble polymers may present miscibility and solubility issues. The polymer hydrogels and water soluble polymers may form solutions, colloidal dispersions, lamelar dispersions or may be fine powder dispersed. To alleviate miscibility or solubility issues, solvents such as ethyl alcohol, acetone and ethyl acetate may be added. Some monomers also behave as solvents, Alternatively, it may be preferable to use lower molecular weight polymer hydrogels and water soluble polymers.

Hydrogels based on natural sources may also be used in the present invention. In general, hydrogels from natural sources can be derived from polymers such as collagen, hyaluronic acid (HA), fibrin, alginate, agarose and chitosan, Depending on their origin and composition, various natural polymers have specific utilities and properties. Many natural polymers, such as collagen, hyaluronic acid, and fibrin, are derived from various components of the mammalian extracellular matrix. Collagen is the main protein of the mammalian extracellular matrix, while HA is a polysaccharide that is found in nearly all animal tissues. Alternatively, alginate and agarose are polysaccharides that are derived from marine algae sources. The advantages of natural polymers include low toxicity and biocompatibility. The disadvantage is that they are relatively weak compared to polymer hydrogels. Collagen and other mammalian-derived protein-based polymers are effective matrices for cellular growth because they contain many cell-signaling domains present in the in vivo extracellular matrix. Collagen gels can be created through natural means without chemical modifications. However, in many cases these gels are mechanically weak.

To synthesize hydrogels with enhanced mechanical properties, various methods have been developed such as chemical crosslinking, crosslinking with UV or temperature, or mixing with other polymeric agents. Collagen degradation is mediated through natural means by proteins such as collagenase. Hyaluronic acid (HA) is a glycosaminoglycan (GAG) that is composed of repeating disaccharide units and is particularly prevalent during wound healing and in joints. Covalently crosslinked HA hydrogels can be formed by means of multiple chemical modifications. HA is degraded by cells through the release of enzymes such as hyaluronidase.

As an alternative to hydrogels, natural and synthetic water soluble polymers may be advantageously used for the same purpose in the present invention. Preferred water soluble polymers include water soluble poly (2-oxazoline), with poly (2-ethyl-2-oxazline) being most preferable. Poly (2-ethyl-2-oxazline) is preferred as a water soluble polymer because, like hydrogels, it creates increased biocompatibility, breatheability and degradability (sloughing) of the cured UV adhesive.

Poly(2-oxazoline) and related water soluble polymers (mostly N-substituted polypeptides, so-called polypeptoids) are emerging as advanced synthetic biomaterials because they are quite readily available, their chemical structures and physical properties can be precisely controlled and adjusted, and they have excellent biocompatibility because they are water soluble. These materials were shown to have great potential for usage especially in biomedical and life science applications. In fact, hydrophilic poly(2-oxazoline) is emerging as a substitute for poly(ethylene glycol), P EG, the “gold” standard in biomedical applications. However, only poly(2-ethyl-2-oxazoline) has been approved by the Food and Drug Administration (FDA), but just as a food contact agent; which is currently the main limitation for wide-spread industrial research on poly(2-oxazoline) based biomaterials and therapeutics. The cationic ring-opening polymerization of 2-oxazolines was discovered in the middle of the 1960s by four independent research groups. The resulting polyamides can be regarded as analogues of poly (amino acids), as shown in the following:

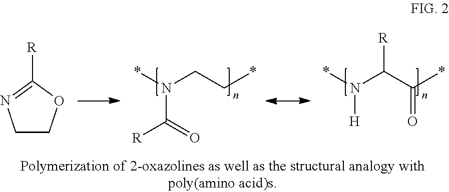

The living cationic ring-opening polymerization of 2-oxazolines provides easy and direct access to a wide variety of well-defined polymers in which the end-group functionality can be controlled during the initiation and termination steps. Furthermore, the properties of poly(2-oxazoline) can be tuned simply by varying the side chain of the 2-oxazoline monomer. With the synthesis and polymerization in recent years, the use of poly (2-oxazoline) in biomedical applications have evolved as a result of their biocompatibility (as well as their stealth behavior, i.e., avoiding uptake by macrophages), being similar to that of polyethylene oxide (PEO).

Water in polymer hydrogels and water soluble polymers provides a moist environment which is very important to wound healing. Skilled practitioners in the art can control the moist environment of the applied adhesive/sealant cured film of the present invention by varying concentrations of hydrogel, water soluble polymers or blends of them to control the equilibrium water content (EWC) of the cured adhesive/sealant. Polyethylene oxide (PEO), for example, dissolves into a solution above its melting point and then forms an opaque, lamellar dispersion when quenched and an opaque film when cured, By reducing film continuity, breathability of the film can be enhanced. In addition to polymer hydrogels and water soluble polymers, one can also use water soluble monomers that behave as hydrogels when they are polymerized or cross-linked. The choice of physical properties of the oligomers and monomers will also impact the equilibrium water content by the level of hydrophobicity/hydrophilicity induced by the oligomers and monomers. Other property enhancing additives will influence the equilibrium water content in the same manner, Equilibrium water content and the film integrity of the cured adhesive determine the water vapor transmission rate of the films formed by the instant invention. Water vapor transmission rate (WVTR) or moisture vapor transmission rate (MVTR) is defined as the quantity of water vapor, under specified temperature and humidity conditions, which passes through a unit area of film material in a fixed time. Water vapor transmission rate (WVTR) is measured in grams per square meter (g/m²) over a 24 hours' period according to US standard ASTM E96-95. It is inversely proportional to the moisture retentive nature of a wound dressing (i.e. the wound dressing with lower WVTR will be able to retain wound surface moisture). Typically, a wound dressing material showing WVTR less than 35g/m²/hr. is defined as moisture retentive and helps in rapid healing,

Photoinitiators are used in the present invention to speed and control the process of photopolymerization (i.e., curing). There are two general routes for photo initiation: free radical and ionic. The general process involves doping a batch polymer with small amounts of photoinitiator, followed by selective radiation of light, resulting in a highly cross-linked product. Many of these reactions do not require solvent, which eliminates a termination path via reaction of initiators with solvent and impurities, in addition to decreasing the overall cost. The result of photo-curing is the formation of a thermoset network of polymers through an exothermic heat release. One of the advantages of photo-curing is that it can be done selectively using high energy light sources, for example, lasers. Nonetheless, photoinitiators are typically required because most monomer and oligomer systems are riot readily activated by light. Dual cure methodologies, such as combining a UV photoinitiator with a humidity/moisture cure, may be used in bonding applications where light is shadowed.

Preferred photoinitiators (PIs) for use in the present invention include, but are not limited to, 1-cyclohexyl phenyl ketone, 2,2-dimethoxy-2-phenylacetophenone (DMPA), 2,4,6-trimethylbenzoyl-diphenyl phosphine oxide, ethyl(2,4,6-trimethylbenzoyl)-phenyl phosphinate, 2-(hydroxyethoxy) phenyl-2-methyl-l-propanone (Irgacure®-2959; D2959; 12959), α-Hydroxyketone, 2-Hydroxy-1-(2-hydroxyethoxy), Irgacure 651® (all Irgacures are ™ of BASF), camphor Quinone/amine, where the amine is triethylamine, triethanolamine and ethyl-N,N-dimethylarninobenzoate,

Changes in structural and chemical properties can also be induced internally by chromophores that the monomer or oligomer already possesses, or externally by addition of photosensitive molecules. A chromophore is the part of a molecule responsible for its color. The color arises when a molecule absorbs certain wavelengths of visible light and transmits or reflects others. The chromophore is a region in the molecule where the energy difference between two different molecular orbitals falls within the range of the visible spectrum. Light that hits the chromophore can thus be absorbed by exciting an electron from its ground state into an excited state.

Other materials can be incorporated into the compositions to add beneficial properties such as biocides, fluorescence agents or dyes to aid placement visibility. Also painkiller or anti-allergy additives may be included to ameliorate wound pain. Many modifiers can be added to the compositions of the present invention to enhance other beneficial properties such as making the product dual cure, that is, in addition to UV curing it can also be moisture curing where light cannot reach the adhesive, Plasticizers, solvents, biocides, biocompatablizers, non-reactive polymers, fibrin, albumin and other proteins may also be added. All of these additives are capable of being added without departing from the invention's basic, novel features.

It is contemplated that sterilization of the compositions may be made with standard medical industry methods.

The following example illustrates how a monomer and oligomer of the present invention can be cross-linked with the aid of a photoinitiator. In this case, the monomer is monomeric styrene and the oligomer is oligomeric acrylate:

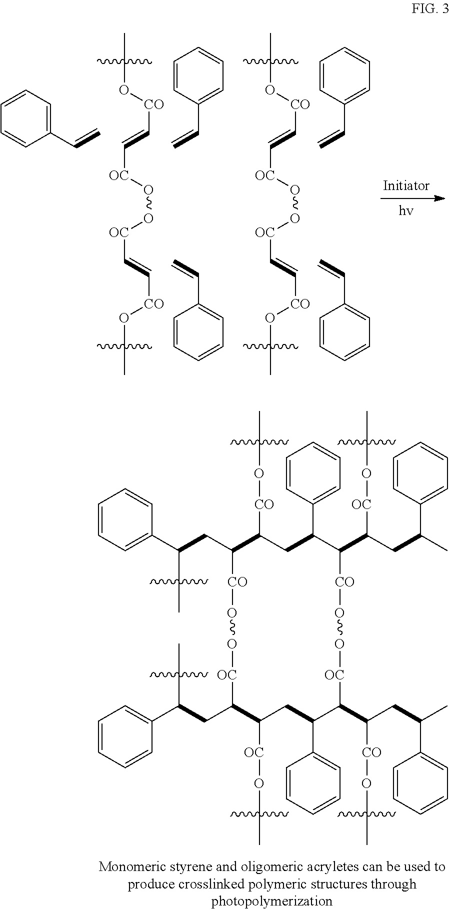

A preferred embodiment of the present invention can be visualized through the following illustration. A liquid adhesive is prepared consisting of suitable monomers, oligomers, hydrogels and/or water soluble polymers and photoinitiator. To seal a wound, a surgeon applies the liquid adhesive to a wound. Application of the liquid adhesive involves dispensing the composition through a light protecting squeeze bottle, squeezable tube, ampule, or syringe type dispenser onto the wound and applying the liquid adhesive where desired. Non-aerosol containers can be used, such as the piston barrier system and the bag-in-can (BOV) system. When the surgeon wants the adhesive to cure, the surgeon or the surgeon's assistant applies UV light to the area where the liquid adhesive solution has been applied. The UV light can come from an UV light generating instrument, such as a penlight, small UV finger squeeze light or optical fiber. The UV light causes the monomers, oligomers and hydrogel/water soluble polymers in the liquid adhesive to quickly cross-link or cure to thereby seal the wound. In the present invention, curing is on demand. In some case, the adhesive cures with surface tack, particularly where solvents are used to act as a temporary plasticizer. Nonetheless, the surface tack in this case disappears when the solvent evaporates from the. cured adhesive. Surface tack can also be removed with a gentle alcohol wipe. The adhesive of the present invention can be supplied as a kit and may advantageously include an isopropanol disposable wipe to cleanse the wound prior to adhesive application.

### EXAMPLES

The following examples are provided to illustrate specific embodiments of the compositions and methods described and should not be construed as limiting the scope of the invention.

### Example 1

A wound closure adhesive was prepared using as the backbone polymer a proprietary compound, Genomer™ 4256, produced by Rahn USA Corp. of Aurora, Ill. Genomer™ 4256 is an aliphatic urethane methacrylate. To prepare the adhesive in this example, a monomer, 2-phenoxyethyl acrylate, was mixed with a water soluble polymer, (200 k Mv) Poly (2-ethyl-2-oxazoline), and a solvent, ethyl alcohol, in the proportions shown below and heated to 80° C. until the water soluble polymer dissolved, The 2-phenoxyethyl acrylate monomer can be obtained from Miwon North America, Inc. of Exton, Pa., the (200 k Mv) Poly (2-ethyl-2-oxazoline) water soluble polymer can be obtained from Polymer Chemistry Innovations, Inc. of Tucson, Ariz. and the ethyl alcohol can be obtained from Consolidated Chemical, Inc. of Allentown, Pa.

| Ingredient | Parts |
|---|---|
| Ethyl alcohol | 1.5 |
| (200k Mv) Poly (2-ethyl-2-oxazoline) | 1.0 |
| 2-phenoxyethyl acrylate | 4.2 |
| Genomer ™ 4256 | 13.1 |
| Ethyl(2,4,6,-trimethyl-benzoyl)phenylphosphinate | 0.9 |
| Total Parts | 20.7 |

Next, the Genomer™ 4256 oligomer was mixed into the composition until a uniform consistency was obtained. The composition was then cooled to approximately 40° C. Finally, the photoinitiator, Ethyl(2,4,6,-trimethyl-benzoyl) phenylphosphinate, was mixed into the composition and the composition was cooled to 30° C. The ethyl(2,4,6,-trimethyl-benzoyl)phenylphosphinate photoinitiator can be obtained from BASF Corp. of Florham, N.J.

0.5 ml of the adhesive composition was applied in liquid form on the under forearm skin of a volunteer. This amount of adhesive is also the amount that a Dermabond™ octyl cyanoacrylate adhesive device expresses onto skin for wound closure. The liquid adhesive composition was cured on the volunteer's forearm by exposing the adhesive to UV 395 mW wavelength penlight for 3 seconds. The adhesive cured to a flexible, clear film with very good skin adhesion and without any feeling by the volunteer of exothermal heat. The applied adhesive withstood a shower within 30 minutes of curing and survived over 6 days of normal activity with no adhesive separation or other disturbance to adhesion. Peripheral edge sloughing was minimally normal each day.

### Example 2

A second wound closure adhesive was prepared which also used Genomer™ 4256 as the backbone oligomer but had a different water soluble polymer. The water soluble polymer in this example was (100 k Mv) Polyethylene oxide) (PEO). PEO can be obtained from Sigma-Aldrich, Inc. of Aurora, Ill. The table below lists the adhesive ingredients and their relative proportions.

| Ingredient | Parts |
|---|---|
| Ethyl alcohol | 1.8 |
| 2-phenoxyethyl acrylate | 3.0 |
| (100k Mv) Poly(ethylene oxide) | 0.75 |
| Genomer ™ 4256 | 12.0 |
| Ethyl(2,4,6,-trimethyl-benzoyl)phenylphosphinate | 0.6 |
| Total Parts | 18.15 |

As in Example 1, the monomer, 2-phenoxyethyl acrylate, was mixed and heated to 80° C. with the water soluble polymer, a powdered, crystalline form of PEO, and a solvent, ethyl alcohol. As in Example 1, the particular monomer was selected because it also acts as a solvent. The Genomer™ 4256 oligomer was added. The composition was mixed until the PEO dissolved. It was observed that the PEO dissolved into the composition at its approximate melting temperature of 67° C. When quenched below that temperature, the PEO became an opaque lamellar dispersion. Finally, the photoinitiator, Ethyl(2,4,6,-trimethyl-benzoyl) phenylphosphinate, was mixed into the composition at 40° C. and the composition was cooled to 30° C.

0.5 ml of the adhesive composition was applied in liquid form on the under forearm skin of a volunteer. The liquid adhesive composition was cured on the volunteer's forearm by again exposing the adhesive to UV 395 mW wavelength penlight for 3 seconds. The adhesive cured to a flexible, opaque film with very good skin adhesion and without any feeling by the volunteer of exothermal heat. The applied adhesive withstood a shower within 30 minutes of curing and survived over 6 days of normal activity with no adhesive separation or other disturbance to adhesion. Peripheral edge sloughing was minimally normal each day.

### Example 3

Example 3 shows the use of polymer hydrogel (PHG) polyethylhexylmethacrylate (PEHMA). This PHG can he obtained from Polymer Chemistry Innovations, Inc. of Tucson, Ariz.

| Ingredient | Parts |
|---|---|
| Ethyl Acetate | 3.0 |
| Ethyl Alcohol | 1.0 |
| 2-phenoxyethyl acrylate | 5.0 |
| Polyethylhexylmethacrylate | 0.8 |
| Genomer 4256 | 10.4 |
| Ethyl(2,4,6,-trimethyl-benzoyl) phenylphosphinate | 0.7 |
| Total Parts | 20.9 |

The ethyl acetate, ethyl alcohol, monomer and PEHMA were mixed at 75° C. until the PEHMA dissolved. Next, the Genomer 4256 oligomer was added and mixed into the composition until a uniform consistency was obtained. The composition was then cooled to approximately 40° C. Finally, the photoinitiator, ethyl(2,4,6,-trimethyl-benzoyl) phenylphosphinate was mixed into the composition and the composition was cooled to 30° C.

0.5 ml of the adhesive composition was applied in liquid form to the under forearm skin of a volunteer. The liquid adhesive composition was cured on the volunteer's skin again, as in Examples 1 and 2, by exposing the adhesive to UV 395 mW wavelength penlight for 3 seconds. The adhesive cured to a flexible, clear film with very good skin adhesion and without any feeling by the volunteer of exothermal heat. As in the previous examples, the adhesive withstood a shower within 30 minutes with no adhesive separation or other disturbance to adhesion. Again, peripheral edge sloughing was minimally normal each day.

The foregoing description set forth preferred embodiments of the invention at the present time. Various modifications, additions and alternative designs will, of course, become apparent to those skilled in the art in light of the foregoing teachings without departing from the scope of the invention. The scope of the invention is indicated by the following claims rather than by the foregoing description. All changes and variations that fall within the meaning and range of equivalency of the claims are to be embraced within their scope.

What is claimed is:

1. An adhesive composition comprising:
an oligomer:
a hydrogel and/or water soluble polymer; and,
a photoinitiator
wherein said adhesive composition will cure when it is exposed to ultraviolet light.

2. The adhesive composition of claim 1 wherein there is curing on demand,

3. The adhesive composition of claim 1 wherein said oligomer is selected from the group consisting of epoxides, urethanes, polyethers, polyesters or a combination thereof.

4. The adhesive composition of claim 1 further comprising a chromophore, a fluorescence agent, a biocide, a painkiller, an anti-allergy agent and/or a plasticizer.

5. The adhesive composition of claim 1 wherein said hydrogel and/or water soluble polymer is polyethylene oxide).

6. The adhesive composition of claim 1 wherein said hydrogel is a natural polymer hydrogel derived from collagen, hyaluronic acid, fibrin, alginate, agarose and/or chitosan.

7. The adhesive composition of claim 1 wherein said photoinitiator is selected from the group consisting of 1-cyclohexyl phenyl ketone, 2,2-dimethoxy-2-phenylacetophenone (DMPA), 2,4,6-trimethyibenzoyl-diphenyl phosphine oxide, ethyl phosphinate, 2-(hydroxyethoxy) phenyl-2-methyl-l-propanone (Irgacure®-2959; D2959; 12959), α-Hydroxyketone, 2-Hydroxy-144-(2-hydroxyethoxy), camphor Quinone/amine, where the amine is triethylamine, triethanolamine and ethyl-N,N-dimethylaminobenzoate,

8. An adhesive composition comprising:
an oligomer:
a hydrogel and/or water soluble polymer;
a monomer; and,
a photoinitiator
wherein said adhesive composition will cure when it is exposed to ultraviolet light.

9. The adhesive composition of claim 8 wherein said oligomer is selected from the group consisting of epoxides, urethanes, polyethers, polyesters or a combination thereof. 10, The adhesive composition of claim 8 wherein said monomer is selected from the group consisting of N-vinylpyrrolidone, acrylates or a combination thereof.

11. The adhesive composition of claim 8 wherein said oligomer has hydrogel and/or water soluble polymer functionality as a hydrogel and/or water soluble polymer.

12. The adhesive composition of claim 8 wherein said monomer is used as a precursor to form said hydrogel and/or water soluble polymer.
